(19) United States
(12) Patent Application Publication
(10) Pub. No.: US 2014/0378555 A1
(43) Pub. Date: Dec. 25, 2014

(54) FACIAL TREATMENT DEVICE

(71) Applicant: AESTHETIC MEDICAL DEVICES LLC, Sherman Oaks, CA (US)

(72) Inventors: George Hung, Sherman Oaks, CA (US); Wayne Chung, Pittsburgh, PA (US); Holly Hung, Sherman Oaks, CA (US)

(21) Appl. No.: 14/370,935

(22) PCT Filed: Jan. 4, 2013
(86) PCT No.: PCT/US13/20385

§ 371 Date: Jul. 7, 2014

Related U.S. Application Data
(60) Provisional application No. 61/583,739, filed on Jan. 6, 2012.

(51) Int. Cl. A61H 23/00; A61K 47/42; A61N 1/32; A61N 1/36; A61F 7/00

(52) U.S. Cl. CPC A61H 23/00; A61N 1/36014; A61F 7/00; A61N 1/325; A61K 47/42; A61H 2201/10; A61H 2201/0207; A61H 2201/105
USPC 514/773; 601/21

(57) ABSTRACT

Embodiments of the present disclosure are directed to portable facial treatment devices, and methods of using same. In some embodiments, the invention is directed to a muscle toning treatment device, comprising: (i) a polarity generator; (ii) a single active electrode assembly comprising a first electrode being adapted to apply electrical current to the area of the skin to be treated; (iii) at least one vibration modality, wherein the vibration modality provides a vibration frequency, of between 5-6 MHz or between 30-35 Hz; (iv) at least one heating modality for heating the skin of the user, wherein the temperature of the heating modality is between 34 and 45 degrees Celsius; and (v) a counter electrode assembly comprising second electrode being adapted to be in continuous contact to a second location of the skin of the user.

### FIELD OF THE DISCLOSURE

Embodiments of the present disclosure are directed to portable facial treatment devices, and methods of using same.

### BACKGROUND OF THE DISCLOSURE

Multiple portable facial treatment devices are known in the art. These devices employ different physical and/or chemical approaches to threat the facial skin and/or the underlying tissue layers, including the facial muscles. Some of these devices are applied directly, while the others assist in delivery and/or absorbance of the serums to the skin.

The known devices deliver electrical energy at different frequencies, heat energy, optical illumination energy, infra-red energy, and ultrasonic energy. The delivered energy acts via physical and/or physiological mechanisms, for example by applying mechanical massage, activating and strengthening the muscles, and improving the blood circulation. The devices can also improve delivery of serum using iontophoresis, or enhancing the skin permeability by ultrasound, or other modalities.

The known in the art portable facial treatment devices deliver the electrical stimulation to the skin using two or more electrodes in contact with the facial skin. The user usually handles the entire the device or part of the device containing the electrodes. The user moves the electrodes over the different areas of the skin in order to deliver the treatment to the entire targeted area. While moving the electrodes, the skin is exposed alternatively, and in an unpredicted manner to anodic or cathodic stimulation. In some cases the treatment requires exposure to only anodic (or only cathodic) stimulation. For example, iontophoresis applies electric field which drives the charged molecules of serum into the skin. Reversing polarity of the stimulation is undesirable, since the reverse filed will push the serum molecules in the opposite direction, thus having detrimental effect on the therapy.

Throughout this description, including the foregoing description of related art, any and all publicly available documents described herein, including any and all U.S. patents, are specifically incorporated by reference herein in their entirety. The foregoing description of related art is not intended in any way as an admission that any of the documents described therein, including pending United States patent applications, are prior art to embodiments of the present disclosure. Moreover, the description herein of any disadvantages associated with the described products, methods, and/or apparatus, is not intended to limit the disclosed embodiments. Indeed, embodiments of the present disclosure may include certain features of the described products, methods, and/or apparatus without suffering from their described disadvantages.

### SUMMARY OF THE DISCLOSURE

According to some embodiments, there is provided, an muscle toning treatment device, comprising: a polarity generator; a single active electrode assembly comprising a first electrode being adapted to apply electrical current to the area of the skin to be treated; at least one vibration modality, wherein the vibration modality provides a vibration frequency of between 5-6 MHz or between 30-35 Hz; at least one heating modality for heating the skin of the user, wherein the temperature of the heating modality is between 34 and 45 degrees Celsius; and a counter electrode assembly comprising second electrode being adapted to be in continuous contact to a second location of the skin of the user.

According to some embodiments, there is provided a second muscle toning treatment device comprising: a polarity generator two concentric electrodes, where the smaller (central) electrode is the cathode, and the larger (external ring) electrode is the return (anode) or vice versa; at least one vibration modality, wherein the vibration modality provides a vibration frequency of between 5-6 MHz or between 30-35 Hz; at least one heating modality for heating the skin of the user, wherein the temperature of the heating modality is between 34 and 45 degrees Celsius.

According to some embodiments, there is provided a muscle toning treatment device, comprising: a polarity generator; an array of active electrode assemblies comprising a first electrode being adapted to apply electrical current, to the area of the skin to be treated at least one vibration modality, wherein the vibration modality provides a vibration frequency of between 5-6 MHz or between 30-35 Hz at least one heating modality for heating the skin of the user, wherein the temperature of the heating modality is between 34 and 45 degrees Celsius; and at least one counter electrode assembly comprising second electrode being adapted to be in continuous contact to a second location of the skin of the user.

The muscle toning treatment devices of the present embodiments may further comprise iontophoresis modality.

According to some embodiments, there is provided a highly charged proteoglycan in combination with a target therapeutic for improved skin health and beauty.

In some embodiments, the the first electrode can be of any shape and the second electrode encircles the first electrode, where both the first and the second electrodes are in contact with the skin during the treatment.

In some embodiments, the first and/or the second electrode are comprised of separate electrically conductive segments. In some embodiments, the second location is the head, neck, shoulder, or hand of the user. In some embodiments, the device emits current in the range of 1-400 μa. In some embodiments, the device provides electrical stimulation below sensory and/or motor threshold. In some embodiments, the device provides electrical stimulation above sensory and/or motor threshold.

The muscle toning treatment devices of the present embodiments may further comprise a means for measuring skin properties selected from the group consisting of temperature, electrical impedance, and mechanical impedance.

The muscle toning treatment devices of the present embodiments may further comprise a means for adjusting the amplitude of the electrical stimulation. The muscle toning treatment devices of the present embodiments may further comprise a means for adjusting the waveform of the electrical stimulation. The muscle toning treatment devices of the present embodiments may further comprise a means for adjusting the frequency of the electrical stimulation. In some embodiments, the device provides a stimulation sequence comprising series of positive pulses alternated with a sequence of negative pulses such that the overall stimulation is balanced. In some embodiments, the stimulation sequence incorporates a gradual increase in the intensity when alternating polarity.

### BRIEF DESCRIPTION OF THE DRAWINGS

For a better understanding of the present invention, reference is made to the following description, taken in conjunction with the accompanying drawings, in which like reference characters refer to like parts throughout.

FIG. 1A schematically illustrates the treatment device in contact with the skin.

FIG. 1B schematically illustrates the treatment device in contact with the skin, with a cable between two modules of the treatment device.

FIG. 2 schematically illustrates the two electrodes (anode and cathode) in contact with the skin, placed at different skin locations.

FIG. 3 schematically illustrates the treatment device with one electrode in contact with the facial skin, and the return electrode located on the device handle.

FIG. 4, Left blank intentionally.

FIG. 5 schematically illustrates the treatment device with one electrode in contact with the facial skin, and the return electrode located elsewhere on the body.

FIG. 6A schematically illustrates the treatment device with multiple electrodes in contact with the facial skin, and the return electrode located on the device handle.

FIG. 6B schematically illustrates the treatment device with multiple electrodes in contact with the facial skin, and multiple return electrodes located on the handle and elsewhere on the body.

FIG. 6C schematically illustrates additional embodiment of the treatment device with multiple electrodes in contact with the facial skin, and multiple return electrodes located on the handle and elsewhere on the body.

FIG. 6D schematically illustrates the treatment device with different shapes of the electrodes in contact with the facial skin. The inner electrode (cathode) is either fully or partially encircled with the outer electrode (anode).

FIG. 7 schematically illustrates the treatment device being used by a person

FIG. 8 schematically illustrates electrodes of the treatment device with heating/cooling/vibrating/ultrasonic elements.

FIG. 9 schematically illustrates electrode of the treatment device in communication with heating/cooling/vibrating ultrasonic elements.

FIG. 10 thru FIG. 25 show examples of the different designs of the treatment device.

FIGS. 26 and 27 schematically illustrate use of stimulating contacts for charging the device battery.

FIG. 28 schematically illustrates use of wireless charger for charging of the device battery.

FIG. 29a shows monophasic stimulation sequences with and without ramp-up period.

FIG. 29b shows biphasic stimulation sequence having a ramp-up period.

### DETAILED DESCRIPTION OF THE INVENTION

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

### Electrical Stimulation

According to some embodiments, the device uses a single electrode in contact with the facial skin (facial electrode). The return electrode may be located far from the facial electrode. Examples of possible embodiments include return electrode located in the handle of the therapeutic device, or attached to the skin (e.g. gel electrode) at the shoulder. Using single electrode resolves the problem of applying consistent polarity to different areas of the facial skin.

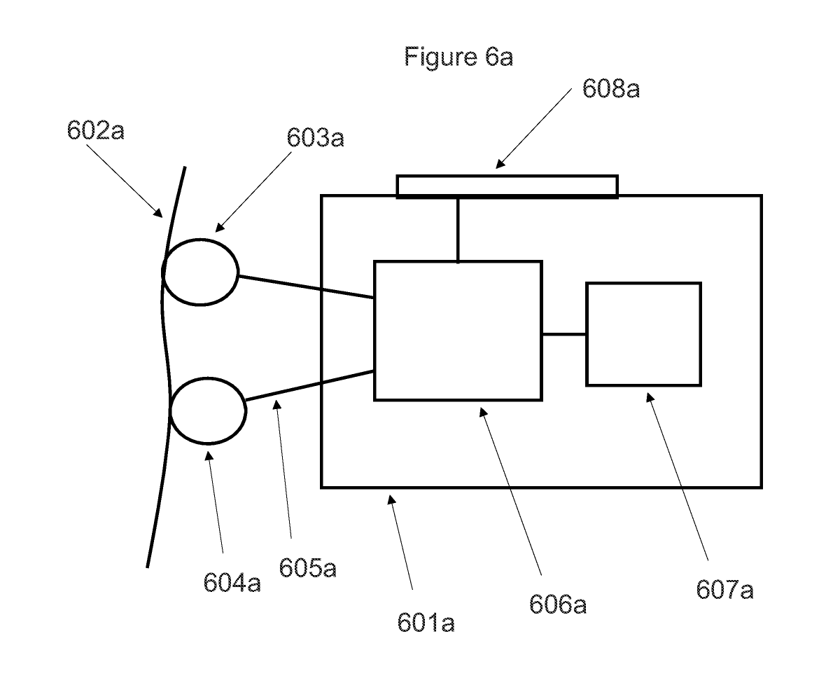

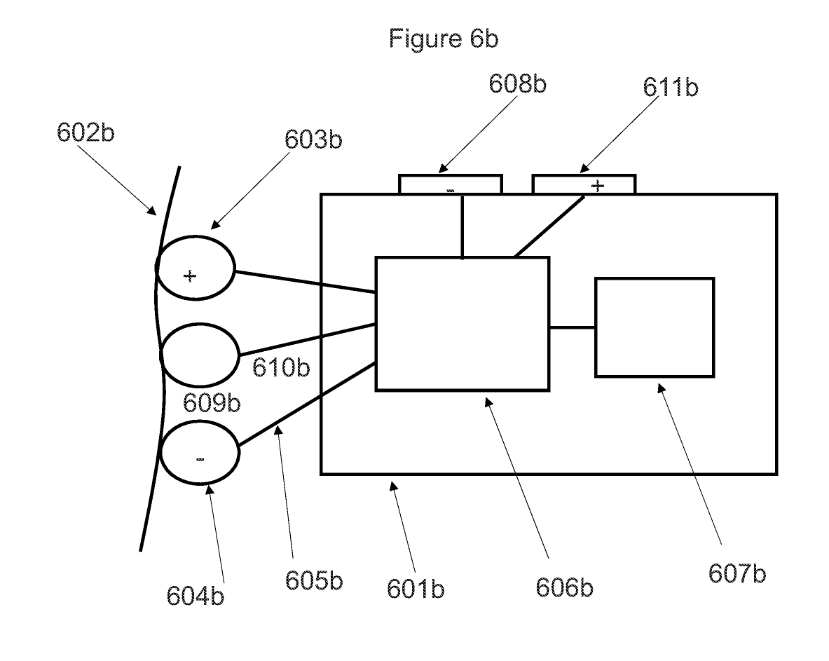

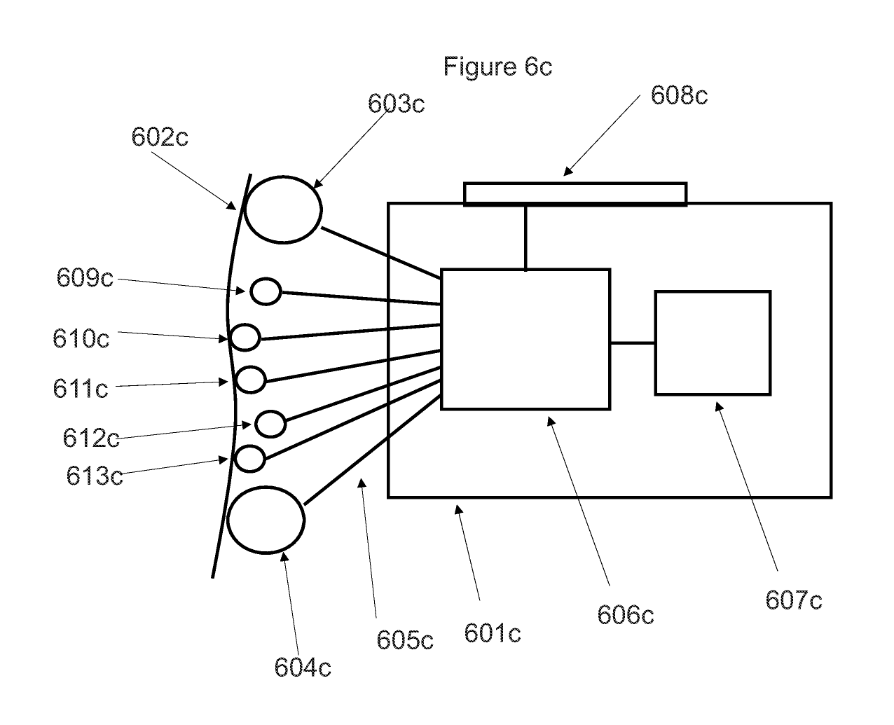

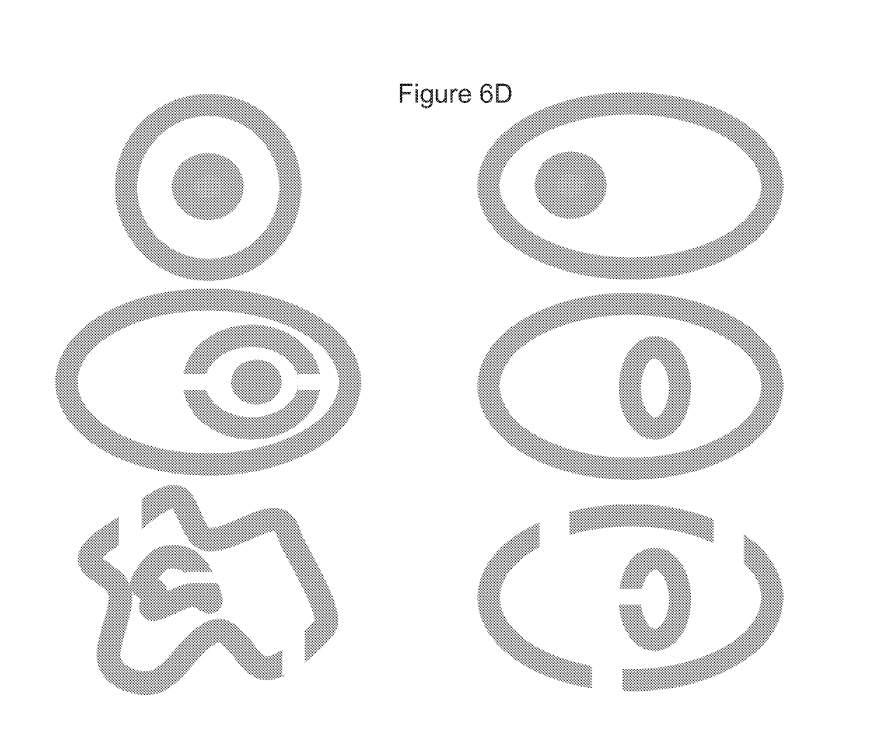

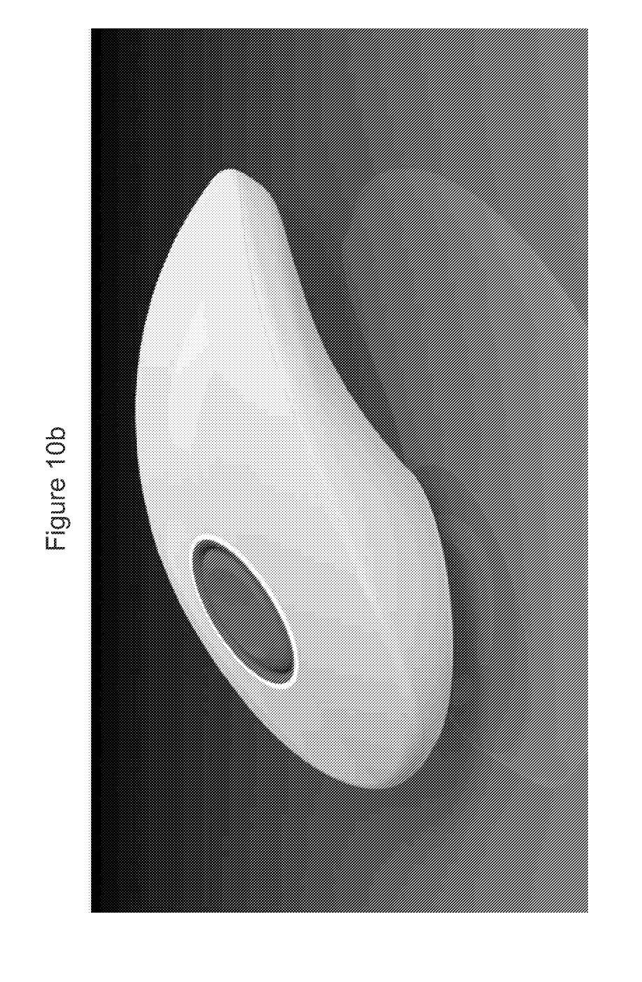

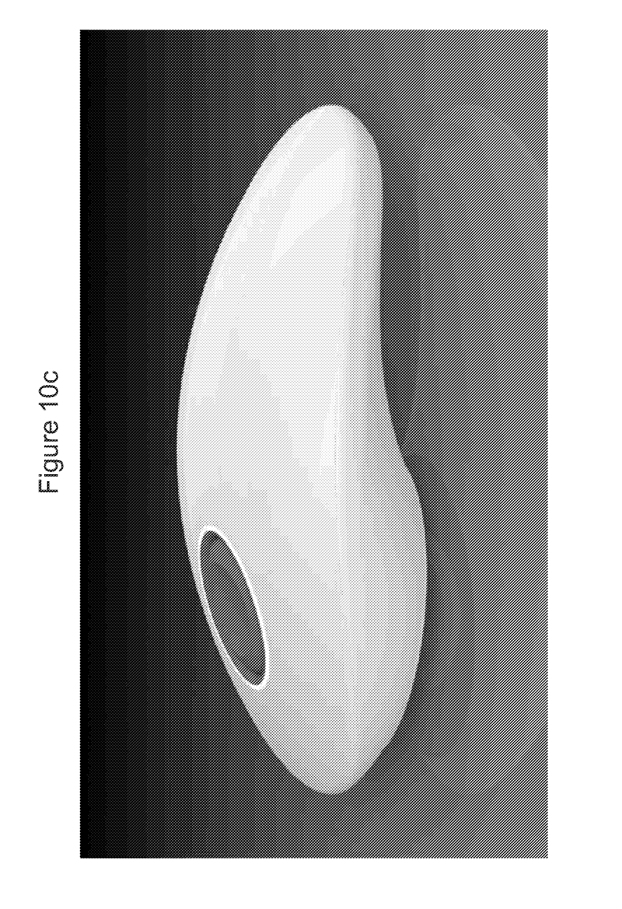

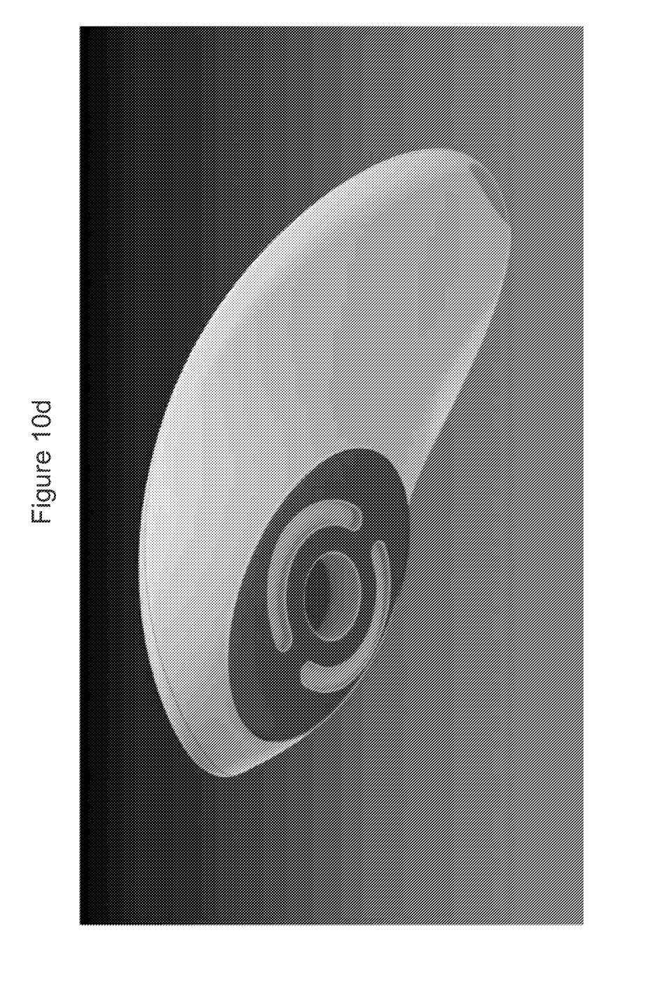

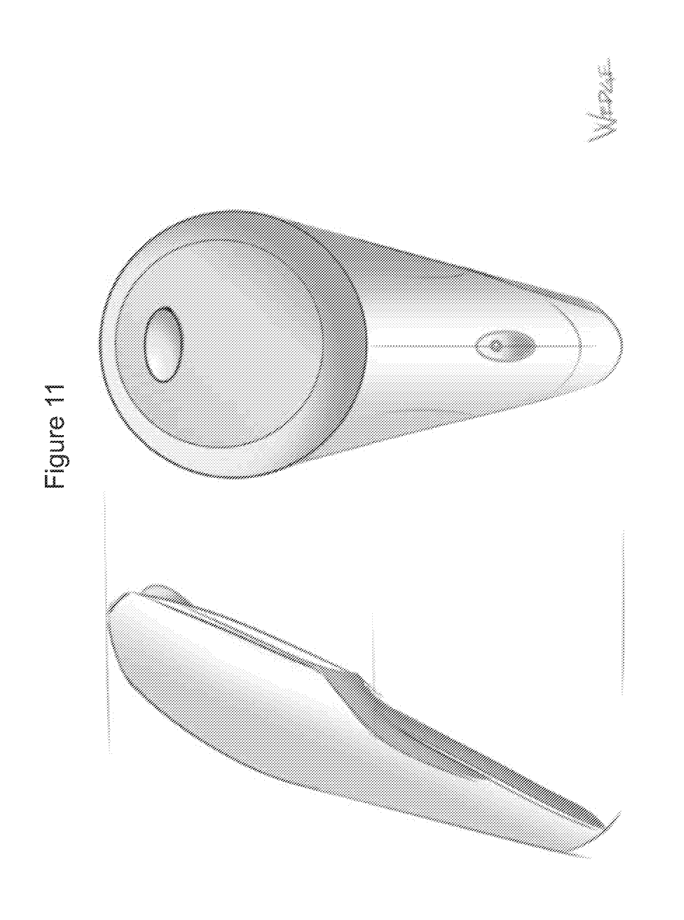

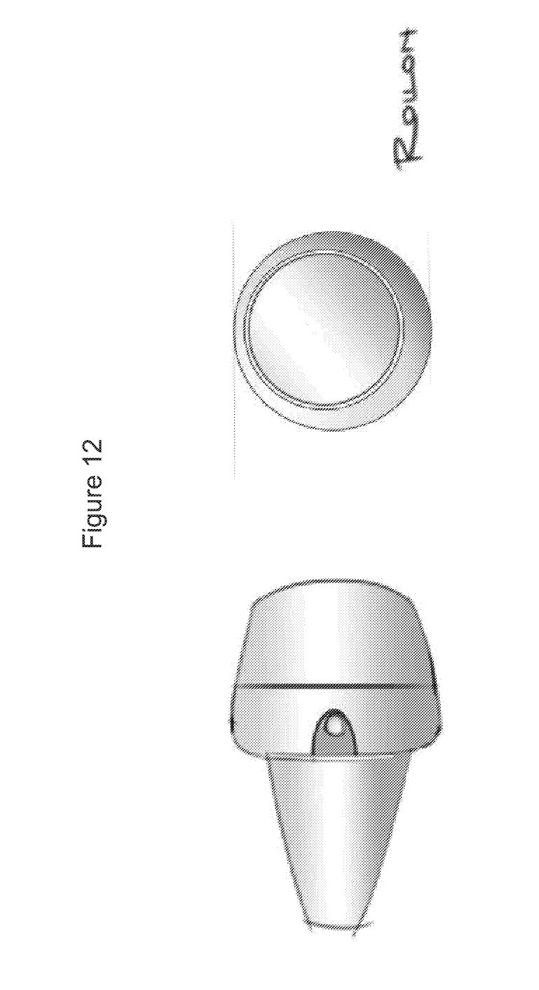

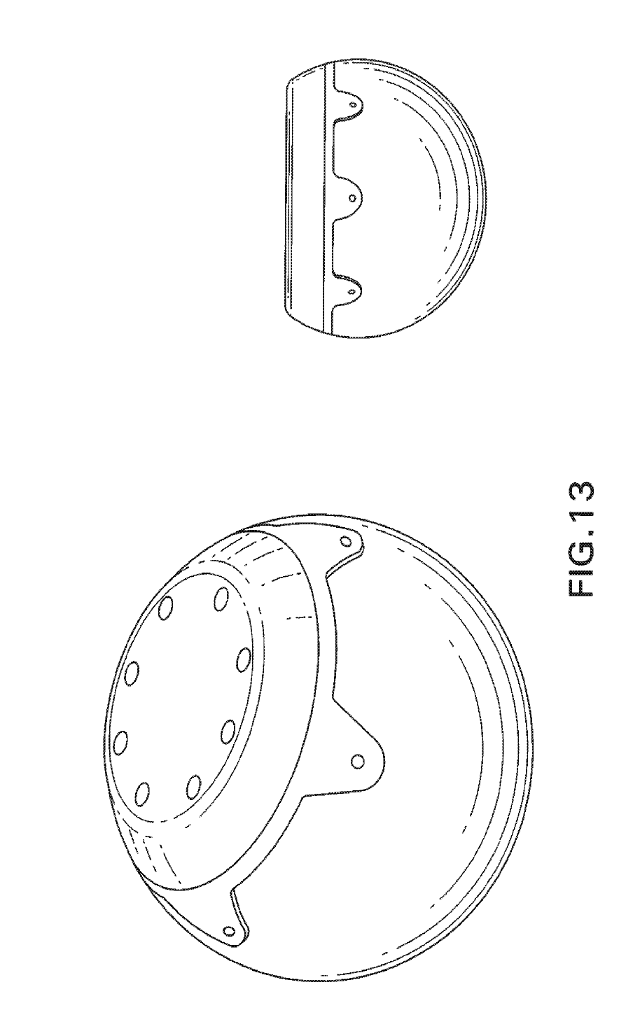

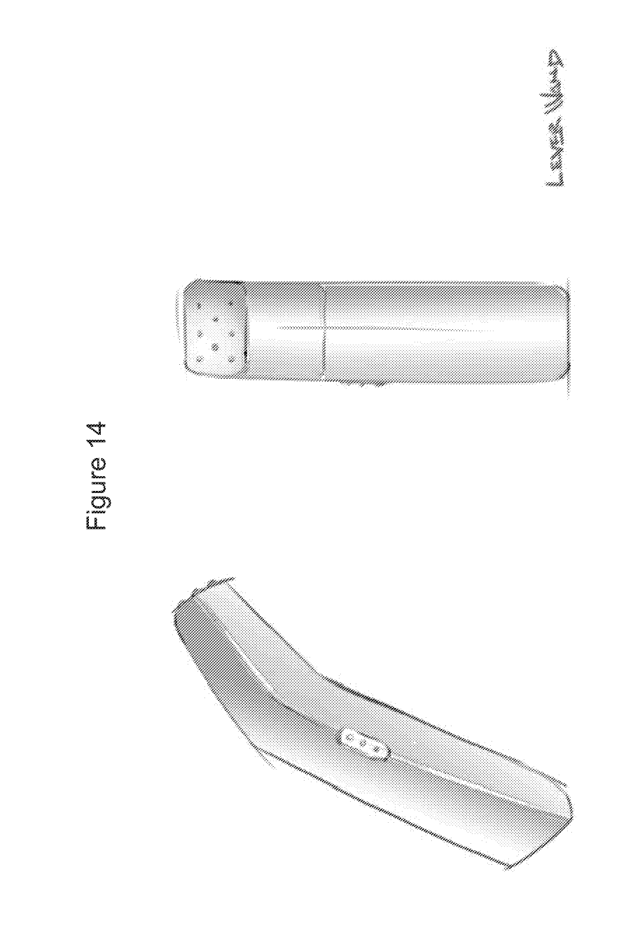

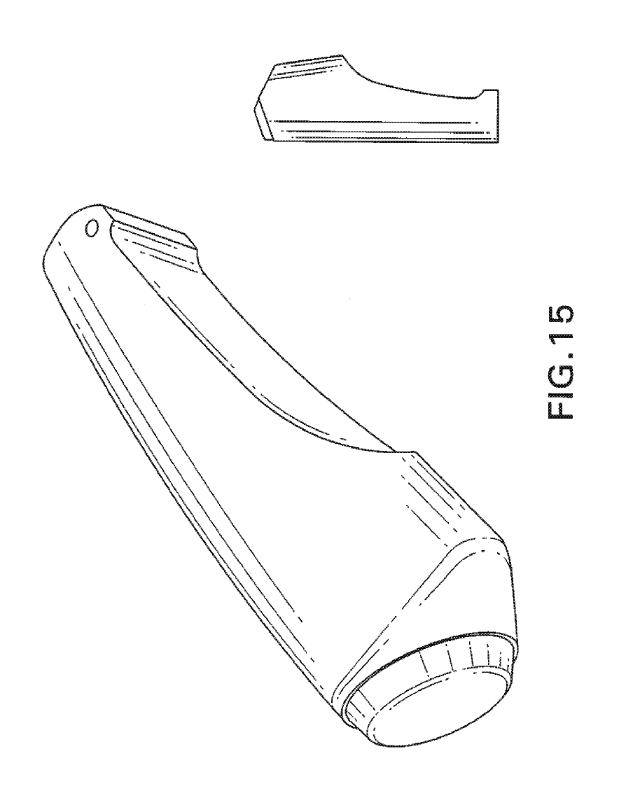

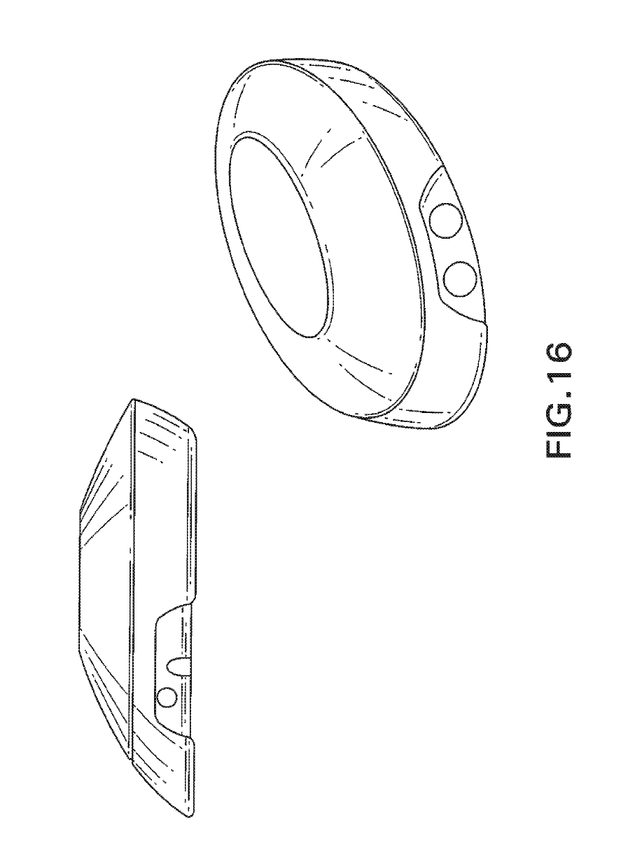

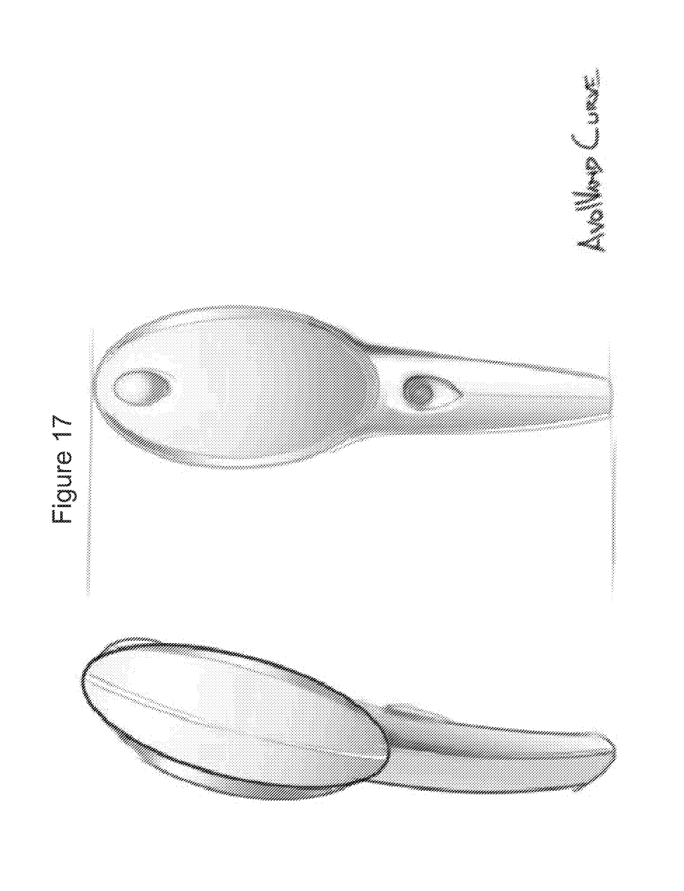

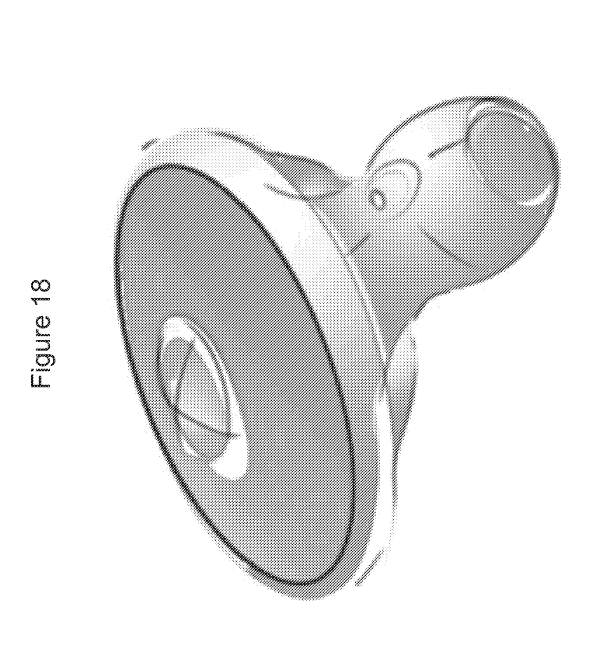

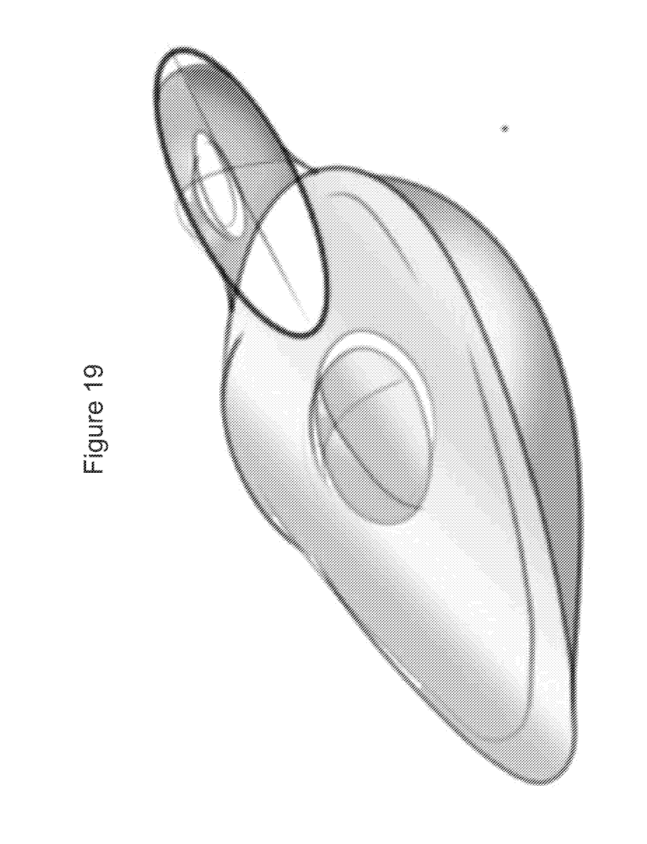

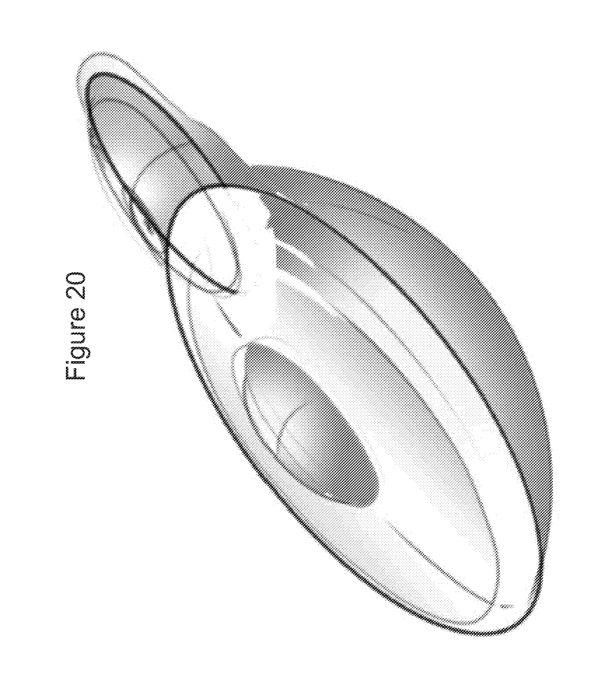

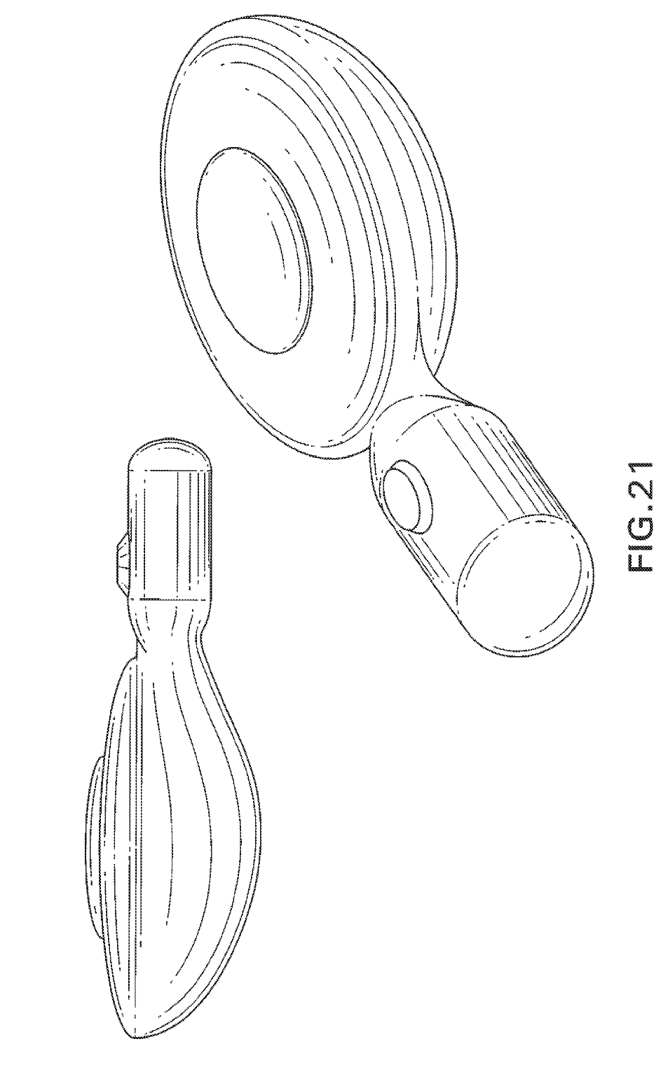

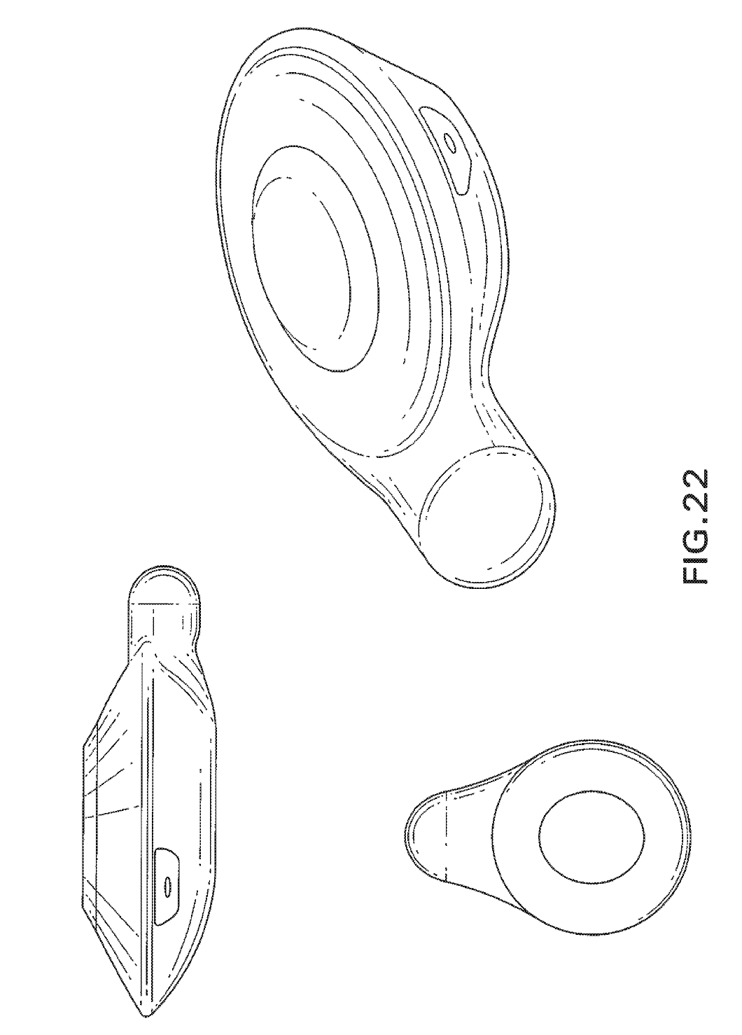

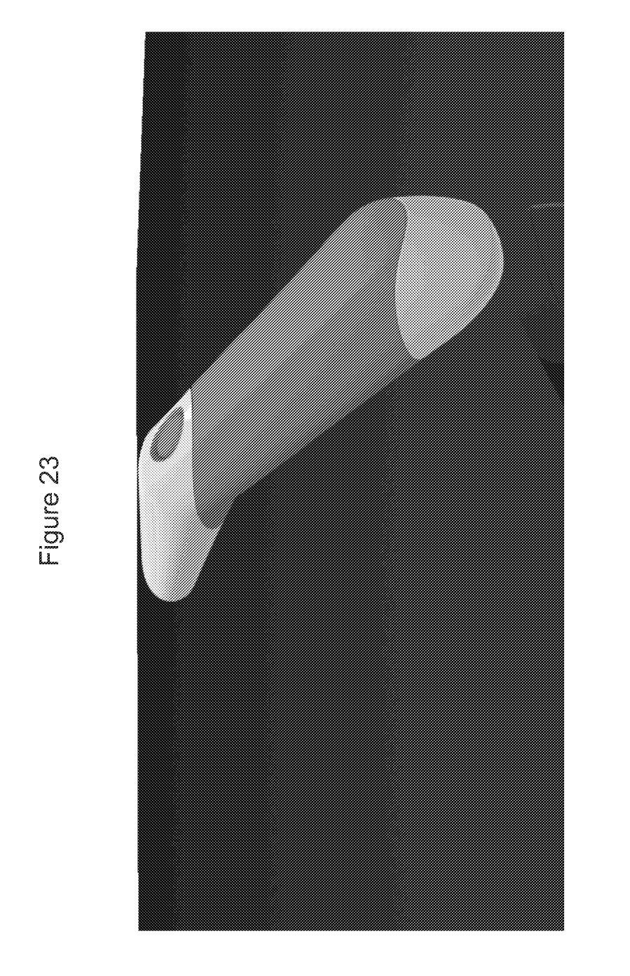

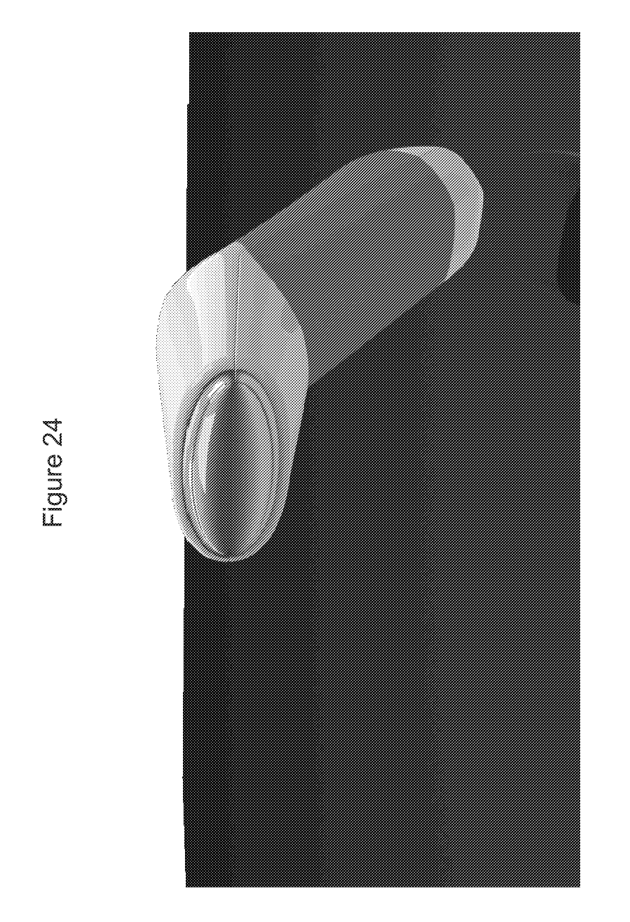

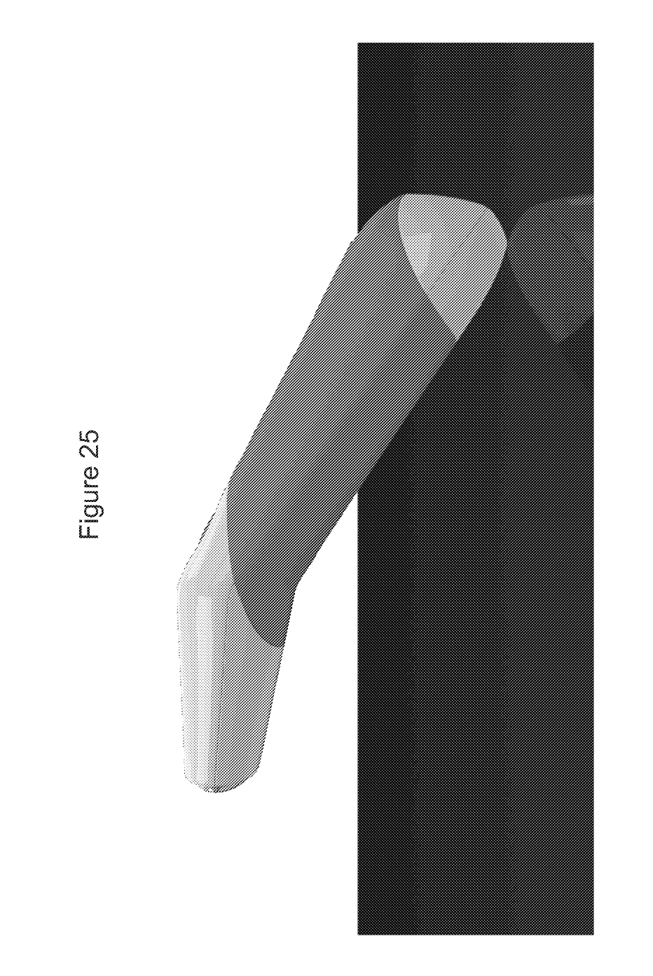

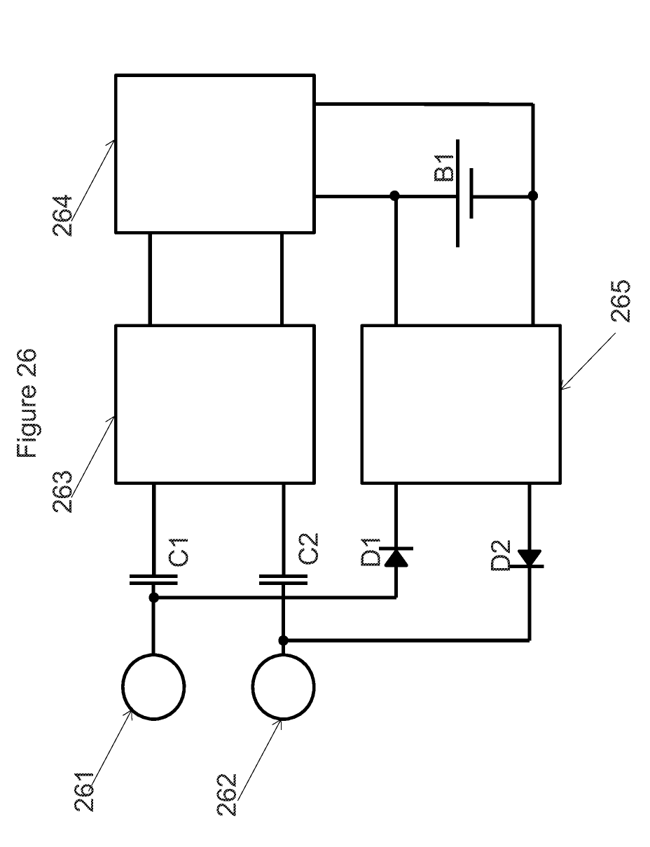

Instead of using a single electrode a ring of concentric electrodes may be used as the primary contact with the facial skin. We propose to have two concentric electrodes, where the smaller (central) electrode is the cathode, and the larger (external ring) electrode is the return (anode). The purpose of this design is to optimally target specific muscles for toning and/or drug delivery. (See FIG. 10a). It should be noted that other designs, not concentric and/or not circular can be also considered. These designs can include elliptical, oval, or other shapes. Moreover, the inner electrode may have one shape, while the outer electrode may have another shape. Each electrode can be comprised of discrete parts, rather than to be made of a single conductive part. Examples of different embodiments are shown in FIG. 6D.

Instead of using a single electrode, array of multiple electrodes may be engaged in contact with the facial skin. Also an army of electrodes may be in contact with the facial skin. The array may be pre-set or changed dynamically to be any combination of anodes and cathodes. The electrode located in the handle or elsewhere on the body is considered to be part of the array and can be programmed to function as anode or cathode in combination with the rest of facial electrodes.

Temperature treatment may be used to affect the efficiency of delivery of serum into the tissue. For example heating the targeted location may improve transdermal permeability due to the combined effect of increased mobility of the serum molecules and to the increased gaps between the dermal cells. Cooling the targeted delivery site will have an opposite effect.

Selective heating/cooling of anode/cathode to promote/reduce the skin permeability.

It should be also noted that although facial treatment is the focus of the examples disclosed herein, the invention can be applied to any other body parts (e.g., neck, arms, back, etc.)

The electrode can be made of metal, cloth, gel, or any other conductive material.

In some embodiments, the device may be used to provide electrical stimulation of different intensity, and waveforms, and frequencies. In some embodiments, the device may be used to provide electrical stimulation having ramp-up period. In some embodiments, the device may be use an array of electrodes for delivery of the stimulation.

In some embodiments, the device may additionally comprise electrodes on device handle.

In some embodiments, the device may comprise an array of electrodes to measure electrical properties of the skin and of the tissues.

In some embodiments, the device may be used to provide electrical stimulation with combination of temperature profile (heating/cooling) and/or mechanical vibrations and/or ultrasound.

In some embodiments, the device may be used to provide stimulation at sub threshold levels (below sensory and motor threshold), or above the sensory threshold, or above the motor threshold.

In some embodiments, the device may be used in a method for enhancing the dermal and transdermal delivery of materials (drug and/or serum).

In some embodiments, the device may be used in combination with other treatments, including Botox treatments.

In some embodiments, the device may be used in a method for stimulating delivery contacts for connection of the device battery charger.

### Heating

According to some embodiments, there is provided a multi-modal energy-based device to provide a combination of electrical stimulation, heating and/or vibration, in any combination thereof. In some embodiments, the target devices include both muscle toning and iontophoresis modalities.

In some embodiments, the device comprises a heating modality. In some embodiments, the temperature of the heating modality is between 34 and 45 degrees Celsius. In some embodiments, the temperature of the heating modality is between 40 and 45 degrees Celsius, preferably 42 degrees Celsius based on US FDA guidance for heated devices that come into contact with the skin. However, we may choose to select a higher temperature of 44 or 45 degrees Celsius if research supports the improved efficacy of 44 or 45 degrees Celsius over 42 degrees Celsius. In some embodiments, the device allows the user to adjust the temperature within the targeted range, such that the temperature to adjust the temperature within the targeted range.

| Category | Fahrenheit | Celsius | Source |
|---|---|---|---|
| Cool Skin | 89.6 | 32 | Internet references. |
| Temperature | | | |
| Normal Skin | 93.2 | 34 | Internet references. |
| Temperature | | | |
| Warm Skin | 96.8 | 36 | Internet references. |
| Temperature | | | |
| Inflammed Skin | 107.6 | 42 | Collagen of chronically inflamed |
| Temperature | | | skin is over-modified and |
| | | | upregulates secretion of matrix |
| | | | metaloproteinase 2 and matrix |
| | | | degrading enzymes by endothelial |
| | | | cells and fibroblasts. Note: based |
| | | | on FDA guidance, 42 degrees may |
| | | | be the final device |
| | | | maximal temperature. |
| Maximum blook | 111.2 | 44 | The effect of increasing |
| flow | | | temperature on skin blood |
| temperature | | | flow and red cell deformability. |
| Theoretical skin | 118.4 | 48 | The temperature of the |
| temperature to | | | applied part should not |
| max | | | exceed 40° C. if the part |
| capillary blood | | | will be in contact with the |
| flow | | | patient for time duration between |
| | | | 1 minute and 10 minutes. |
| Maximum | 123.8 | 51 | Internet references. |
| Massage Stones | | | |
| Causes bum in | 140 | 60 | Reducing the incidence |
| 5-6 seconds | | | of tap-water scalds: |
| | | | strategies for physicians. |
| Causes bum in 2 | 150.8 | 66 | Reducing the incidence of |
| seconds | | | tap-water scalds: |
| | | | strategies for physicians. |
| Causes burn in 1 | 158 | 70 | Reducing the incidence of |
| second | | | tap-water scalds: |
| | | | strategies for physicians. |

### Vibration

In some embodiments, the device comprises a vibration modality. In some embodiments, the device may comprise a range of vibrations between a lower vibration and higher vibration. Lower vibrations may be suitable for user sensory feedback and thereby enhance user compliance. Higher vibration levels are suitable to offer more benefit to therapy. Low frequency can be also used for massage. We believe both modalities will be critical for optimal device results.

For therapeutic vibration levels, the vibration modality may provide a range of between 1-5 MHz. For sensory feedback, the vibration modality may provide a range of between 30-35 Hz, which is thought to be suitable to enhance user compliance.

Additional frequency range that can be used for massage is within hundreds of Hz, for example 100-500 Hz.

| Speed | | Device |
|---|---|---|
| 30-35 | Hz | Whole body vibration-Beginner |
| 35-40 | Hz | Whole body vibration-Intermediate |
| 40-50 | Hz | Whole body vibration-High |
| 1 | MHz | Ultrasound Massager HS3008 |
| 5 | MHz | Ultrasound Massager SR-06A |
| 5 | MHz | Hitachi Magic Want Vibrating Massager |
| 6 | MHz | Hitachi Magic Want Vibrating Massager |
| 24 | MHz | Bio-Therapeutics Microdermabrasion |

Iontophoresis

Electromotive Drug Administration (EMDA), or Iontophoresis, is a technique using an electric charge to deliver a medicine, salts, chemical or water through the skin. It is essentially a transdermal delivery system that does not require a needle. The goal of iontophoresis is deeper, more rapid delivery of a therapeutic through the skin. The technical description of this process is a non-invasive method of propelling high concentrations of a charged substance, normally a medication or bioactive agent, transdermally by repulsive electromotive force using a small electrical charge applied to an iontophoretic chamber containing a similarly charged active agent and its vehicle.

According to one aspect, the present invention relates to the development of endogenous charged proteoglycan to serve as a delivery vehicle for iontophoresis. In some embodiments, the selected proteoglycan is one that occurs during inflammation or as part of a natural healing process. One such example is heparin. Heparin is also known as unfractionated heparin, a highly sulfated glycosaminoglycan. Heparin is a naturally occurring protein and it is widely used as an injectable anticoagulant. Heparin has one of the highest negatively charge density of any known biological molecule. Cox, M.; Nelson D. (2004). Lehninger, Principles of Biochemistry. Freeman. p. 1100, ISBN 0-71674339-6. The benefit of having a proteoglycan that is highly charges is that if it can be complexed with a therapeutic target, it will be possible to deliver heparin and the target payload with iontophoresis. Other proteoglycans that we have identified include: heparin, keratin, chondroitin, dernatan and hyaluronic acid. See also, Kristian Prydz and Knut Tomas Dalen. Synthesis and sorting of proteoglycans. Journal of Cell Science 113, 193-205 (2000), incorporated herein by reference in its entirety.

According to another aspect, the invention relates to combining a charged delivery system with a target therapeutic that could be used to improve overall skin health. In some embodiments, the target therapeutic may include, but is not limited to, one or more of the following: Gd contrast Agents, TGF-Beta, Retinoic Acid (Vitamin A), Oestrogen, ACAT-1 Inhibitor K-604, Angiotensin II, Velvet Antler Extract, Duabanga grandiflora leaf extract, Alpha-D-glucosylglycerol, Ascorbic acid (Vitamin C), Alpha-tocopherol (Vitamin E). In some embodiments, the target therapeutic is one or more NF-kappa beta inhibitors, including beta hydroxybutirate. In some embodiments, the target therapeutic is spinach extract. In some embodiments, the target therapeutic is an anti-oxidant.

According to another aspect, the invention relates to an iontophoresis device. In some embodiments, the iontophoresis device is designed for dermatology delivery of one or more therapeutic agents disclosed herein. In some embodiments, the device may have additional features including vibration and heating to aid delivery and uptake of the target therapeutic. In some embodiments, the iontophoresis device may include at least one charged electrode on the delivery area. In the instance of the heparin delivery system, the electrode may be negatively charged to repel the negatively charged heparin system. In some embodiments, the electrode may be a positively charged vesicle. In some embodiments, the positively charged electrode repels the positively charged vesicle for deeper penetration into stratum corneum, dermal and subdermal tissue.

In some embodiments, an iontophoresis device includes of DC voltage delivery system and electrodes. Wires may then connect between the unit and the active and passive electrodes, and the unit is set for specified current and defined amount of time. In the iontophoresis process, the current may be transferred from the electrode through the ionized drug solution as ionic flow. The drug ions may be moved to the skin where the repulsion continues moving the drug through the trans-appendageal structures and stratum corneum interstices via the aqueous pores. The larger the electrode surface, the greater the current the device must supply to provide a current density for moving the drug or agent. Rai R, Srinivas C R. Iontophoresis in dermatology. Department of Dermatology, PSG Hospitals, Peelamedu, Coimbatore, India.

Iontophoresis may be used to deliver lidocaine or other anesthetics, such as Iontocaine, a local anesthetic with vasoconstrictor, administered via iontophoresis through the skin. Iontocaine can numb up to 10 mm of skin in as quick as 10 minutes (2% lidocaine, 0.01 mg/ml epinephrine solution). Fentanyl is another anesthetic that may be used with iontophoresis.

There are some iontophoresis devices that deliver salts as part of physical rehabilitation. The Trivarion Buffered Iontophoresis Electrode System is one such patch and it is intended for the administration of soluble salts into the body for therapeutic purposes, and as an alternative to hypodermic injections. Banta C. A, A Prospective, Nonrandomized Study of Iontophoresis, Wrist Splinting, and Anti-inflammatory Medication in the Treatment of Early-Mild Carpal Tunnel Syndrome, JOM 1994; Vol 36, No 2; 166-168.

Iontophoresis with water may be used to treat excessive sweating of the hands and feet. In some embodiments, the procedure uses a mild electrical current that is passed through tap water to temporarily shut off sweat glands. A hand and foot is each placed in a different water basin. The electric current is gradually increased to the required level and maintained for 20 minutes before gradually decreasing the current. The procedure is repeated with the other side.

Tap water iontophoresis is generally effective within one month. In one study, 85 percent of patients with excessive perspiration of the palms had normalization of sweating. In another study, patients had an average of 81 percent improvement with treatment. Patients receive three treatments per week until the sweating is controlled (average of 10 treatments). Once sweating normalizes, patients may need as little as one treatment every 24 weeks for maintenance. At that point the patient may buy the machine from the manufacturer with a physician's prescription.

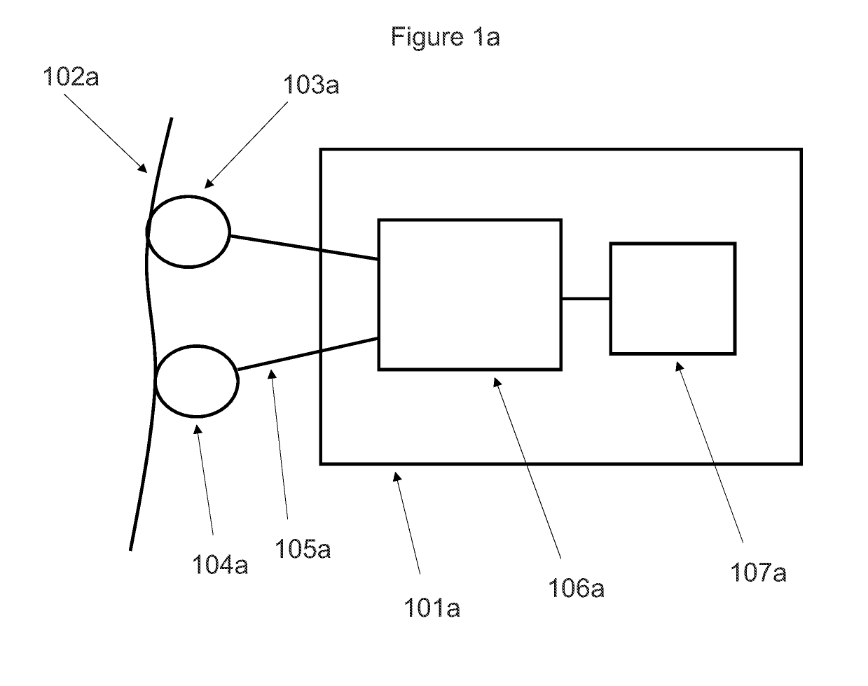

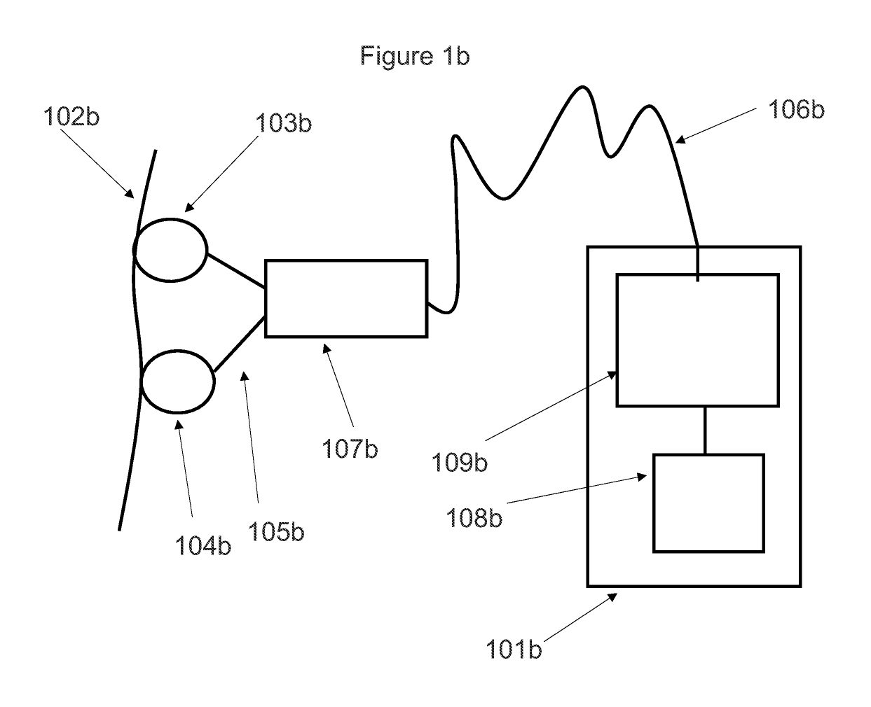

Reference is now made to FIG. 1A. Face treatment device is in contact with the skin (102a), via two electrical contacts (103a and 104a), connected to the device via rigid, flexible, or semi-flexible conductors (105a). The contacts 103a and 104a may be made of metal, conductive gel, wet cloth, or other biocompatible conductive materials. The treatment device includes a generator (106) generating the required waveform to be delivered to the skin. The waveform parameters (shape, frequency, amplitude, and others) may be set via a user interface located on the devices (not shown), or on the remote control (not shown). The waveform parameters can be also adjusted based on a feedback received from measuring the skin properties (temperature, electrical impedance, mechanical impedance, and others). The generator receives the energy from the power supply (107), which may be based on primary or rechargeable batteries, or receive a power from the AC line.

Reference is now made to FIG. 1B, which shows the embodiment similar to that shown in FIG. 1a. The difference is that not all parts of the treatment device are located in the same unit. The electrodes (103b, 104b) are connected to the handle (107b). The handle is connected by the cable (106b) to the remote unit containing the generator (109b) and the power supply (101b). It should be noticed that the arrangement of the modules between the handle and the remote units may be different. For example, the stimulator may be located in the handle, while the power supply is located in the remote unit.

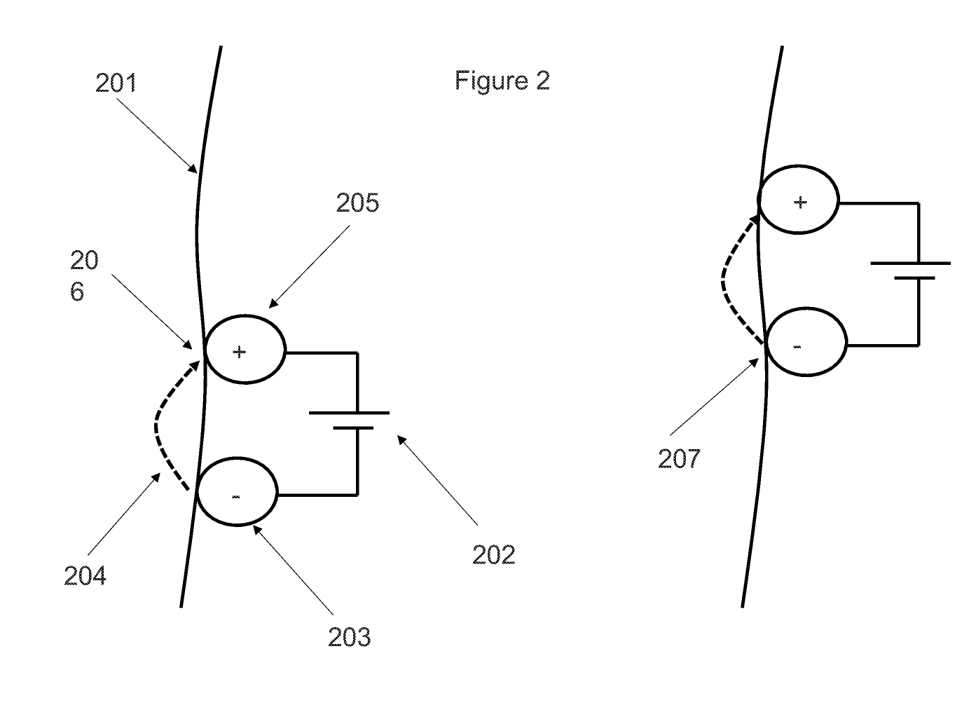

Reference is now made to FIG. 2, which shows two contacts: positive (205) and negative (203) in contact with the skin 201. The polarity generator is schematically shown as a battery 202, but can be comprised of any other functional module. The positive and negative electrodes (cathode and anode) create transcutaneous electric field, schematically shown by a dotted line (204). If the drug/serum containing negative ions will be placed under the positive electrode, these negative ions will be pushed into the skin (tissue) at this location (206). The treatment device, however, usually is moved on the skin in order to treat different areas. The negative electrode may be, therefore, moved to a location that was just treated by a positive electrode (207), thus reversing the movement of the ions, and decreasing the efficiency of the drug/serum delivery.

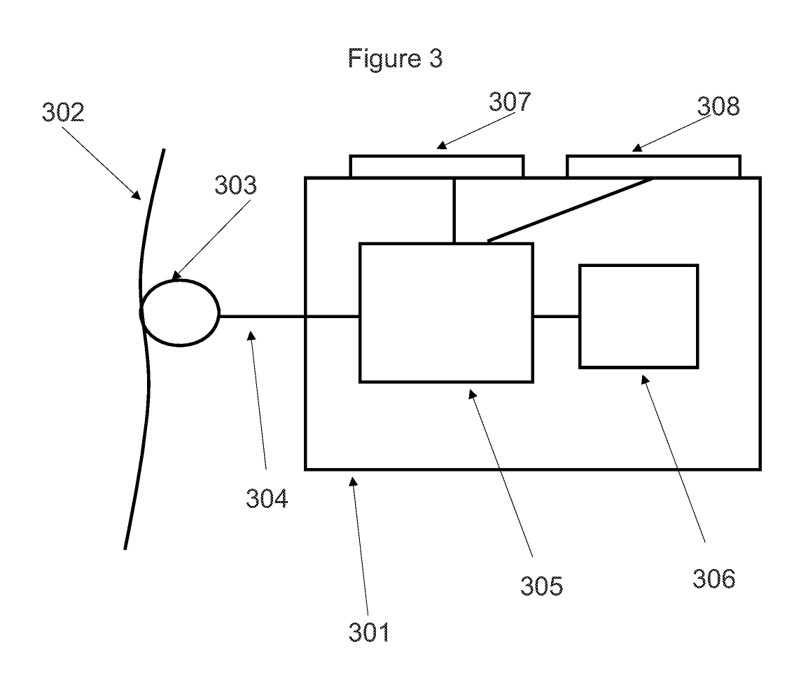

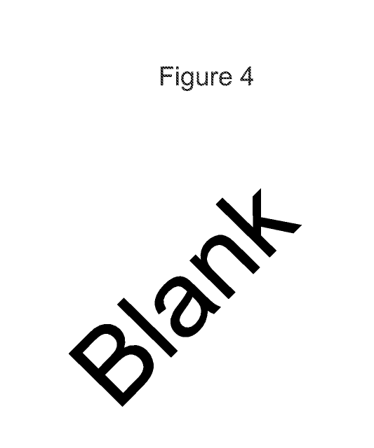

Reference is now made to FIG. 3. One of the electrodes (303) is in contact with the skin (302). The electrode is connected to the stimulating circuits (305) via a conductor (304). The return electrode(s) (307, 308) are located on the handle of the treatment device (301). The device is powered by a power supply (306). If the active electrode (303) is in contact with the face, than the electrical path via the body will include the head, the neck, the shoulder, and the hand. The return electrodes are, therefore, located remotely from the active electrode, resulting in practically monopolar stimulation of the face. In this embodiment the facial skin will be in contact with the active electrode, but never with the return electrode, thus enabling, for example, to apply the same stimulation polarity to the facial skin. The surface of the return electrode(s) is usually significantly larger than the surface of the active electrode in order to achieve low current density at the return electrode, and not causing any physiological effects at the return electrode. The return electrode can be for example, in a shape of conductive ring on the device handle, or in a shape of segmented ring on the handle.

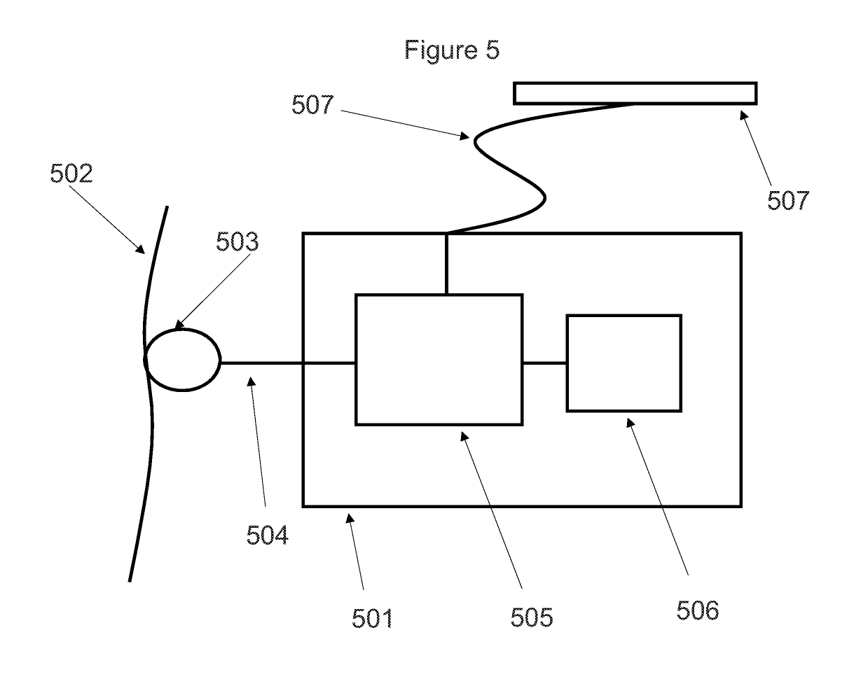

FIG. 5 shows an alternative embodiment, where the return electrode(s) (507) are not located on the device, but are connected to the device via a cable (507). The return electrode(s) can be located elsewhere on the body.

FIG. 6A demonstrates an embodiment with plurality of electrodes (603a and 603b) in contact with the facial skin. FIG. 6b demonstrates that plurality of electrodes can be in contact with the facial skin (603b, 604b, 610b), and plurality of electrodes can be in contact with the hand, or elsewhere on the body (608b, 611b). The electrodes can be arranged in an array. Electrodes' polarity can be dynamically changed, based on pre-set configuration, or on a dynamic allocation, for example 603b and 611b are shown positive, electrodes 604b and 608b are negative, and electrode 609b is shown neutral.

FIG. 6C shows an array of electrodes (609c, 610c, 610c, 611c, 612c, 613c). Due to the curvature of the skin (602c), some of the electrodes can be in full contact with the skin, while the others may be not in contact with the skin, or in partial contact with the skin. Electrical impedance can be measured between different combinations of the electrodes. Since the geometrical relationships between the different electrodes are known, the measured impedances can be normalized. Based on the normalized impedance, characteristic tissue impedance can be calculated. Also, it is possible to estimate which of the electrodes are actually in contact with the skin. Characteristic impedance can be used to monitor, identify, and indicate areas of the skin having different impedance. The areas with different impedance can require additional treatment, for example. Also, knowing the impedance can be used as a safety factor, for example indicating when the electrodes are not in full contact with the skin, and stopping the stimulation, in order to keep low and safe current/charge density via the electrodes.

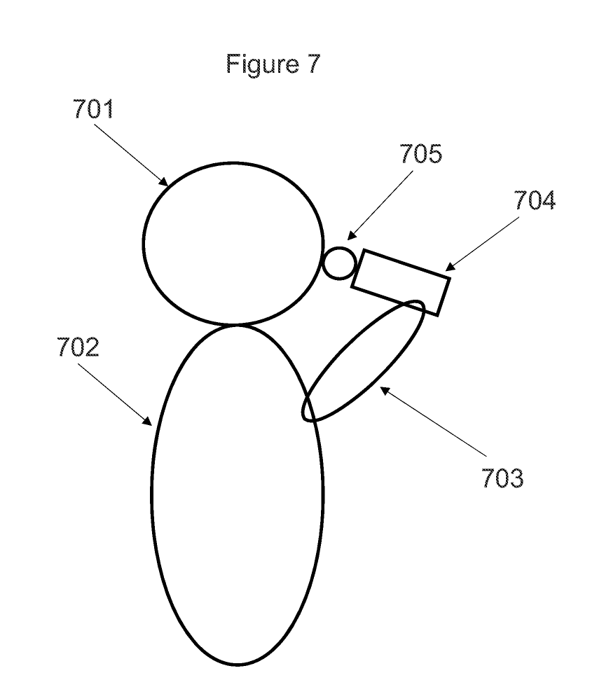

FIG. 7 demonstrates application of the device, where the active electrode (705) is in contact with the face (701). The device is kept by the handle (704), in the hand (703).

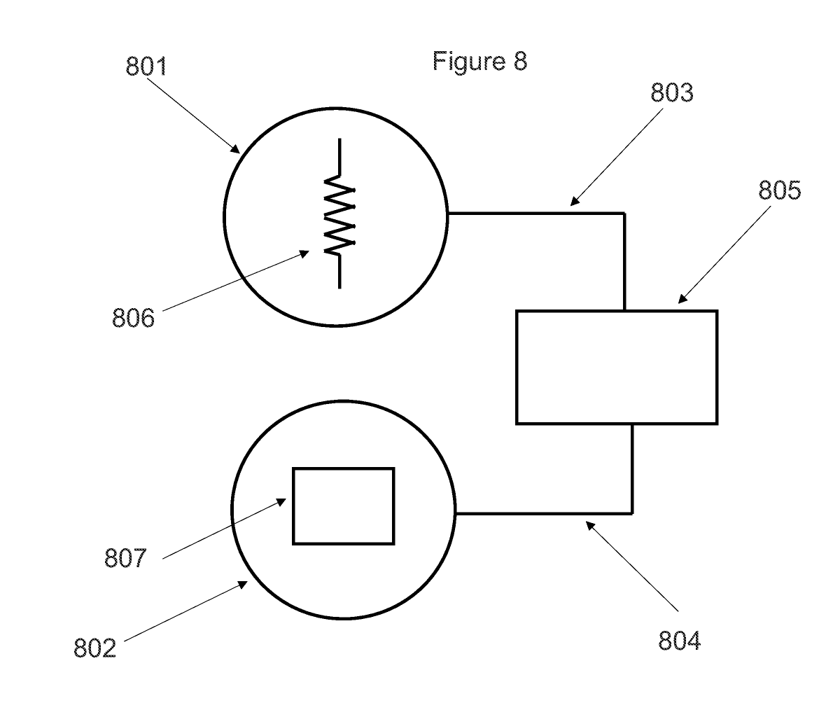

FIG. 8 demonstrates that additional modalities can be combined with the electrical stimulation. For example, heating element (806) can be embedded, attached, or otherwise connected and deliver the heat energy to the electrode (801). Heating profile of the electrode can be coordinated with the profile of electrical stimulation. In addition to heating, temperature profile may include cooling, for example using Peltier cooling element (807). It should be noted that mechanism (802) can also include a mechanism delivering mechanical vibrations to the electrode (massaging or applying ultrasound.

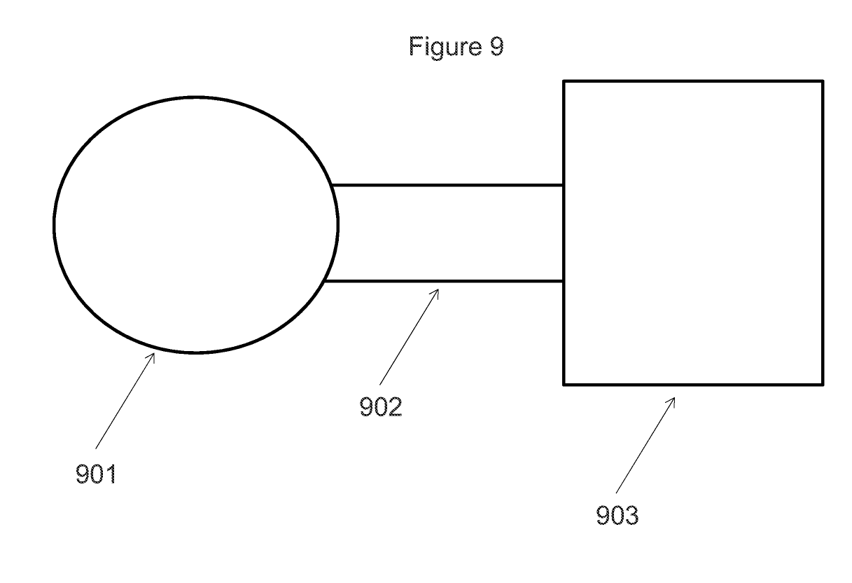

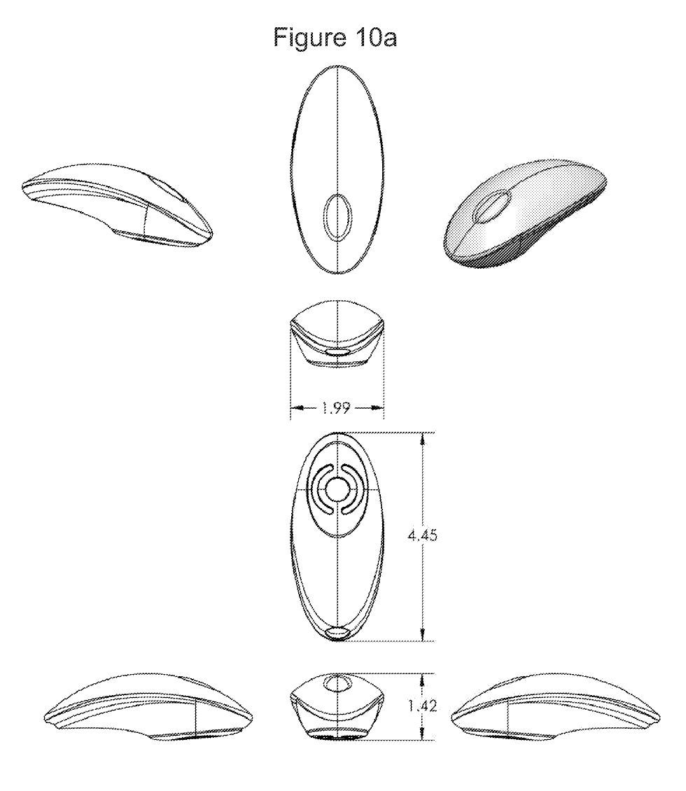

FIG. 9 shows that the heating/cooling/vibrating element can be located outside the electrode (901), and deliver the energy to the electrode via connecting element (902).

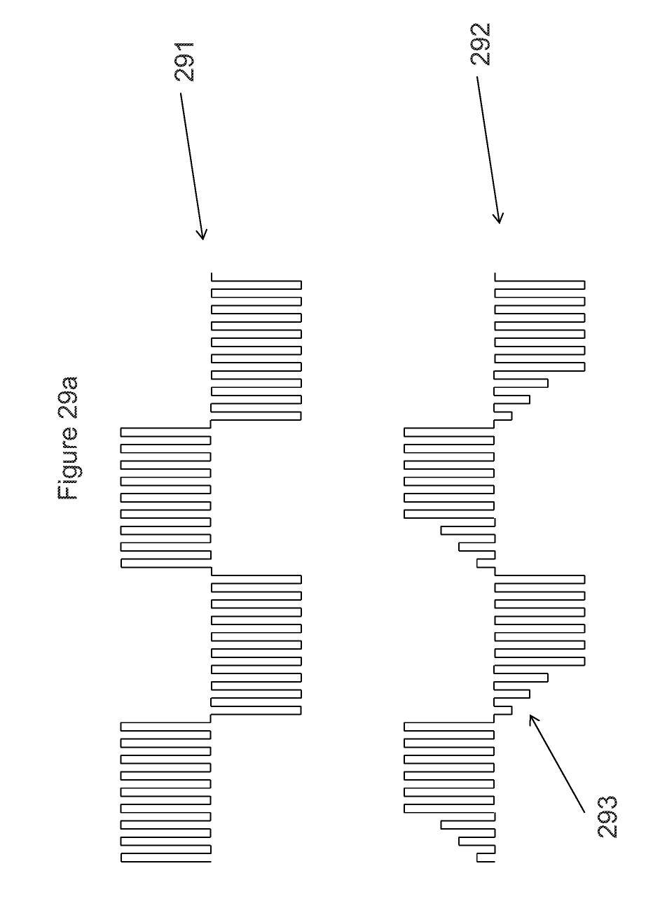

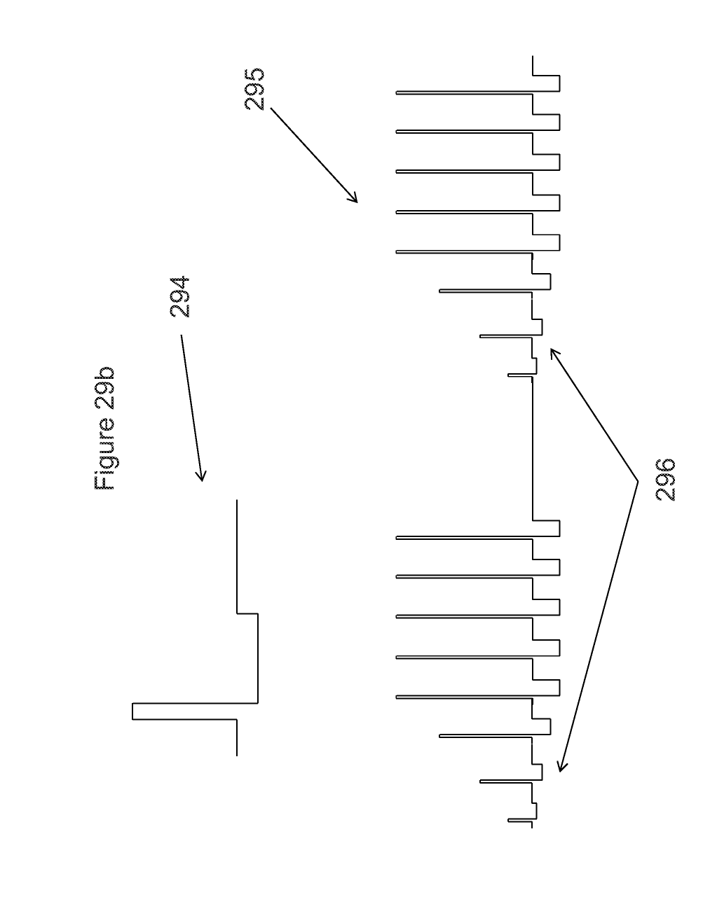

It should be noted that the sensation related to the delivery of electrical stimulation is dependent on the amplitude and other parameters of the stimulation. For example, FIG. 29a shows a sequence of monophasic stimulation (291). A series of positive pulses alternates with a sequence of negative pulses in order to keep the overall stimulation balanced. An abrupt switch from positive stimulation to negative stimulation can cause a strong sensation. It is known in electrical stimulation that gradual increase in the intensity of the stimulation causes milder perception. Stimulation sequence (292) incorporates the gradual increase in the intensity (293), also known as ramp-up period. It should be mentioned that the ramp-up period can be incorporated for various stimulation waveforms, for example for bi-phasic, asymmetrical pulse (294), as shown in FIG. 29b. Here the stimulation sequence (295) includes ramp-up periods (296).

FIG. 10 to FIG. 25 show examples of different designs of the treatment device.

The proposed device can operate using the main power, or it can be battery operated. The battery may be either primary (non-rechargeable) or rechargeable. Recharging the battery requires a charger, which either have galvanic connection to the device, or can engage wireless recharging mode. In order not to add additional charging connector, it is possible to use the same contacts that are used for delivery of the stimulation to the skin. FIG. 6 shows an example of such embodiment. The stimulating electrodes (261, 262) are connected to the stimulus generating circuit (263). The electrodes may be (but do not have to be) AC coupled via the capacitors C1 and C2. When a DC charger is connected to the contacts 261 and 262, the current from the charger (not shown) flows to the charging circuit (265) via the diodes D1 and D2. The charging circuit charges the rechargeable battery B1. When operational, the power supplying module (274) delivers the B1 battery power to all other circuits of the device.

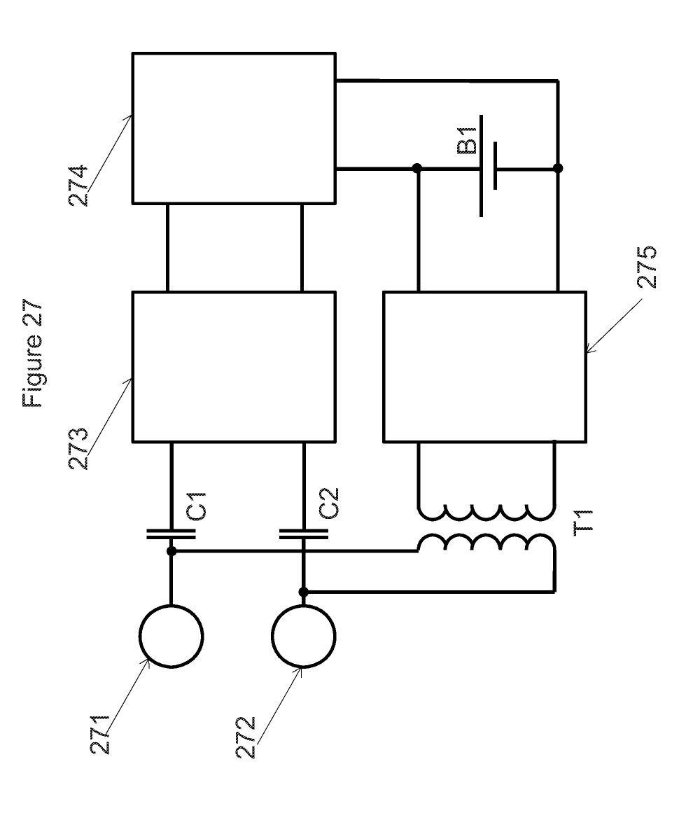

FIG. 27 shows an example of the alternative design of the circuit. The electrical path of the stimulation and the charging is decoupled via the transformer T1. The AC current delivered by the external charger (not shown) to the contacts 271 and 272 is transferred to the charging circuit 275 via the transformer T1.

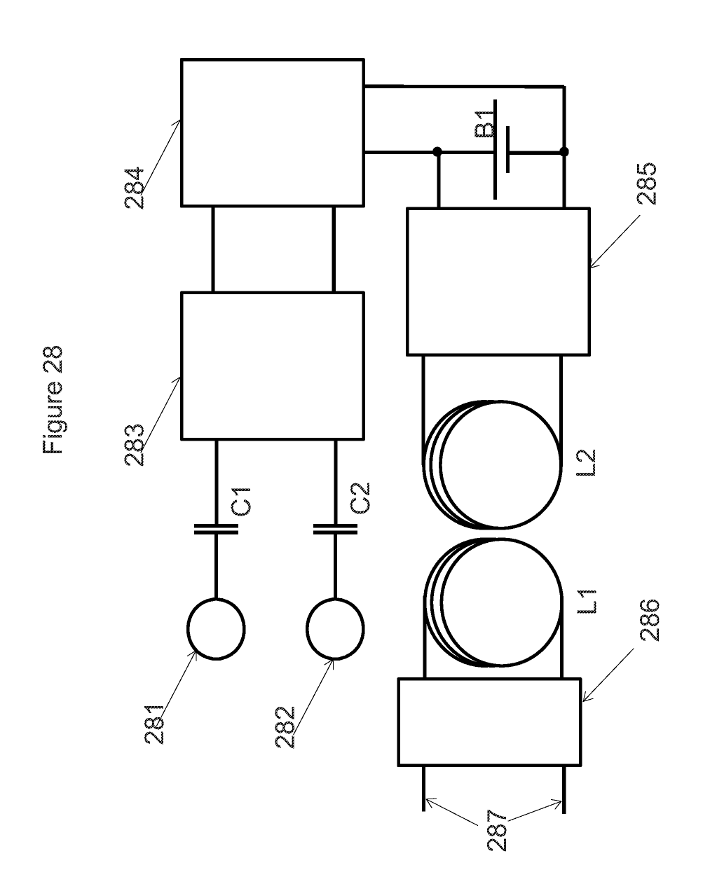

FIG. 28 shows an example of using wireless charger to recharge the device. A charger includes a coil L1 and a coil driver (286). The charger is connected to the primary power via the lines 287. The coil generates an alternating magnetic field, which is received by a coil in the device (L2). The received AC energy is rectified, and used by the charging circuit 285 to charge the battery B1.

In order to prolong the use time of the device battery, it is proposed that the device will identify a “no use” situation, for example when the electrodes are not in touch with the skin for some time. In this case the device can automatically turn itself off.

### Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the invention will be apparent from the following detailed description and claims.

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “an agent” is a reference to one or more agents and equivalents thereof known to those skilled in the art, and so forth. Thus, for example, a reference to “an electrode” includes a plurality of such electrodes.

The terms “proximal” and “distal” will be used to describe the opposing axial ends of the inventive closure device, as well as the axial ends of various component features. The term “proximal” is used in its conventional sense to refer to the end of the apparatus (or component thereof) that is closest to the operator during use of the apparatus. The term “distal” is used in its conventional sense to refer to the end of the apparatus (or component thereof) that is initially inserted into the patient, or that is closest to the patient.

What is claimed is:

1. A muscle toning treatment device, comprising:
i. a polarity generator;
ii. a single active electrode assembly comprising a first electrode being adapted to apply electrical current to the area of the skin to be treated;
iii. at least one vibration modality, wherein the vibration modality provides a vibration frequency of between 5-6 MHz or between 30-35 Hz;
iv. at least one heating modality for heating the skin of the user, wherein the temperature of the heating modality is between 34 and 45 degrees Celsius; and
v. a counter electrode assembly comprising second electrode being adapted to be in continuous contact to a second location of the skin of the user.

2. A muscle toning treatment device comprising:
i. a polarity generator
ii. two concentric electrodes, where the smaller (central) electrode is the cathode, and the larger (external ring) electrode is the return (anode) or vice versa.
iii. at least one vibration modality, wherein the vibration modality provides a vibration frequency of between 5-6 MHz or between 30-35 Hz;
iv. at least one heating modality for heating the skin of the user, wherein the temperature of the heating modality is between 34 and 45 degrees Celsius; and

3. The device of claim 1 or 2, further comprising an iontophoresis modality.

4. The device of any one of claims 1 to 3, wherein the first electrode can be of any shape and the second electrode encircles the first electrode, where both the first and the second electrodes are in contact with the skin during the treatment.

5. The device of any one of claims 1 to 4, where the first and/or the second electrode are comprised of separate electrically conductive segments.

6. The device of any one of claims 1 to 5, wherein the second location is the head, neck, shoulder, or hand of the user.

7. The device of any one of claims 1 to 6, wherein the device emits current in the range of 1-400 μs.

8. The device of any one of claims 1 to 7, wherein the device provides electrical stimulation below sensory and/or motor threshold.

9. The device of any one of claims 1 to 8, wherein the device provide electrical stimulation above sensory and/or motor threshold.

10. The device of any one of claims 1 to 9, further comprising a means for adjusting the amplitude of the electrical stimulation.

11. The device of any one of claims 1 to 10, further comprising a means for adjusting the waveform of the electrical stimulation.

12. The device of any one of claims 1 to 11, further comprising a means for adjusting the frequency of the electrical stimulation.

13. The device of any one of claims 1 to 12, wherein the device provides a stimulation sequence comprising series of positive pulses alternated with a sequence of negative pulses such that the overall stimulation is balanced.

14. The device of any one of claims 1 to 13, wherein the stimulation sequence incorporates a gradual increase in the intensity when alternating polarity.

15. An muscle toning treatment device, comprising:
i. a polarity generator;
ii. an array of active electrode assemblies comprising a first electrode being adapted to apply electrical current to the area of the skin to be treated;
iii. at least one vibration modality, wherein the vibration modality provides a vibration frequency of between 5-6 MHz or between 30-35 Hz;
iv. at least one heating modality for heating the skin of the user, wherein the temperature of the heating modality is between 34 and 45 degrees Celsius; and
v. at least one counter electrode assembly comprising second electrode being adapted to be in continuous contact to a second location of the skin of the user.

16. The device of claim 15, further comprising an iontophoresis modality.

17. A highly charged proteoglycan in combination with a target therapeutic for improved skin health and beauty.

18. A method of improving skin health and beauty in an individual, the method comprising forming a highly charged proteoglycan in combination with a target therapeutic on the individual.
